(19) United States
(12) Patent Application Publication
(10) Pub. No.: US 2009/0016962 A1
(43) Pub. Date: Jan. 15, 2009

(54) COMPOSITIONS AND METHODS FOR THE TREATMENT OF CANCER

(75) Inventors: Dai Fukumura, Newton, MA (US); Lance L. Munn, Lexington, MA (US); Rakesh K. Jain, Wellesley, MA (US)

Correspondence Address: EWARDS ANGELL PALMER & DODGE LLP, P.O. BOX 55874, BOSTON, MA, 02205, US

(73) Assignee: THE GENERAL HOSPITAL CORPORATION, Boston, MA (US)

(21) Appl. No.: 12/067,505

(22) PCT Filed: Oct. 3, 2006
(86) PCT No.: PCT/US06/38680

§ 371 Date: Aug. 22, 2008

Related U.S. Application Data
(60) Provisional application No. 60/723,289, filed on Oct. 3, 2005.

(51) Int. Cl. A61K 9/50; A61K 49/08; A61K 9/127; A61P 35/00

(52) U.S. Cl. 424/9.1; 424/490; 424/450

(57) ABSTRACT

Provided are nanostructures comprising a charged outer surface and an inner core comprising a cancer therapeutic agent or imaging agent, wherein the charged outer surface is selectively removable. Further provided are methods of treating subjects having cell proliferative disorders, e.g., cancer, and kits comprising the above nanostructures.

### RELATED APPLICATIONS

This application claims the benefit of U.S. provisional application Ser. No. 60/723,289, filed Oct. 3, 2005, the entire contents of which are incorporated herein by reference.

### GOVERNMENT SUPPORT

The work leading to the present invention was funded in part by contract/grant numbers CA80124 and CA56591, from the United States National Institutes of Health. Accordingly, the United States Government may have certain rights to this invention.

### INCORPORATION BY REFERENCE

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND OF THE INVENTION

The diagnosis and treatment of solid tumors is notoriously difficult. Among the many problems encountered in the clinical treatment and diagnosis of cancer is the ability of a clinician to administer a cancer therapeutic or diagnostic agent to the site of the tumor. For blood borne-delivery, any therapeutic or diagnostic agent crosses the tumor vessel wall and moves through the intersitital compartment to reach the cancer cells. Tumor vessels are disorganized in their structure and function (Jain, R. K. et al. (2002) Nature Reviews Cancer 2: 266-276). As a result, the chaotic blood supply of a tumor and heterogenous vascular permeability prevent uniform transvascular transport to tumors (Jain, R. K. (1987) Cancer Metastasis Rev. 6: 559-593, Jain, R. K. (1988) Cancer Res., 48: 2641-2658). On the other hand, certain features of tumor vasculature also provide an opportunity for selective targeting (Bremer, C. et al. (2001) Nat Med, 7: 743-748, Campbell, R. B et al. (2002) Cancer Research, 62: 6831-6836, Hobbs, S. K., et al. (1998) PNAS 95: 4607-4612). Once across the tumor vessel wall, the movement of molecules and particles through the interstitium depends on their size, charge and configuration, and the physico-chemical properties of the interstitium. Interstitial hypertension in tumors reduces convection from blood vessels and flushes drugs out of tumors (Jain, R. K. (1987) Cancer Research, 47: 3039-3051, Jain, R. K. (1994) Scientific American, 271: 58-65, Jain, R. K., (2002) Nature Reviews Cancer, 2: 266-276). These barriers hinder uniform delivery of any therapeutic agent to targets within the tumor.

Accordingly, a need exists for effective methods and compositions for targeting therapeutic and diagnostic agents to cells in solid tumors.

### SUMMARY OF THE INVENTION

The instant invention is based, at least in part, on the discovery that particles with cationic charge, e.g., high cationic charge, and larger size will preferentially target tumor vasculature as compared with normal vessels. Based on this observation, the instant invention provides compositions and methods for the treatment of cell proliferative disorders, such as those characterized by solid tumors.

Accordingly, in one aspect the instant invention provides a nanostructure comprising a charged outer surface and an inner core comprising a cancer therapeutic agent or an imaging agent, wherein the charged outer surface is selectively removable. In a related embodiment, the inner core comprises one or more quantum dots, polymers, liposomes, silicon, silica, dendrimers, microbubbles and/or nanoshells. In a specific embodiment, the inner core further comprises one or more quantum dots. In another related embodiment, the quantum dots are CdSe quantum dots.

In another embodiment, the charged outer surface is attached to the inner core by a peptide. In a related embodiment, the peptide is cleavable.

In another embodiment, the inner core is comprised of two or more quantum dots, polymers, liposomes, silicon, silica, dendrimers, microbubbles and/or nanoshells attached to each other by a peptide, such as a cleavable peptide.

In other related embodiments, the peptide is cleavable by a protease, e.g., a protease expressed by a tumor, Cathepsin B, Cathepsin D, MMP-2, Cathepsin K, Prostate-specific antigen, Herpes simplex virus protease, HIV protease, cytomegalovirus protease, thrombin, or interleukin 1β converting enzyme. In a specific embodiment, the protease is MMP-2 and the cleavable peptide comprises the amino acid sequence PLGVRG (SEQ ID NO:1) or PLGLAG (SEQ ID NO:2).

In another embodiment, the outer surface is cationic at physiological pH. In a related embodiment, the charged outer surface is comprised of a material selected from the group consisting of polyethylene glycol (PEG), N-(2-hydroxypropyl)methacrylamide (HPMA), poly(vinyl-pyrrolidone) (PVP), poly(ethyleneimine) (PEI), a polyamidoamine, divinyl ether and maleic anhydride (DIVEMA (DIVEMA), dextran (α-1,6 polyglucose, dextrin (α-1,4 polyglucose), hyaluronic acid, a chitosan, a polyamino acid, poly(lysine) or poly(glutamic acid), poly(malic acid), poly(sapartamides), poly co-polymers, or copaxone. In a specific embodiment, the charged outer surface is comprised of polyethylene glycol (PEG). In a related embodiment, the PEG is derivitized, e.g., to comprise a trimethyl ammonium moiety, a carboxylic acid moiety, a sulfonic acid moiety, or a hydroxyl group.

In another embodiment, the nanostructure is about 10-30 nm in diameter, about 10-50 nm in diameter, about 10-100 nm in diameter, or about 10-400 nm in diameter.

In another embodiment, the inner core comprises a matrix of PEG silicate.

In another aspect, the invention provides a nanostructure comprising a charged outer surface and an inner core comprising a cancer therapeutic agent or an imaging agent in one or more members of the group consisting of quantum dots, polymers, liposomes, silicon, silica, dendrimers, microbubbles and/or nanoshells, wherein the outer surface is attached to the inner core by cleavable peptides.

In a related embodiment, the cleavable peptide is cleavable by a protease, e.g., a protease expressed by a tumor, Cathepsin B, Cathepsin D, MMP-2, Cathepsin K, Prostate-specific antigen, Herpes simplex virus protease, HIV protease, cytomegalovirus protease, thrombin, and interleukin 1β converting enzyme. In a specific embodiment, the protease is MMP-2 and the cleavable peptide comprises the amino acid sequence PLGVRG (SEQ ID NO: 1) or PLGLAG (SEQ ID NO:2).

In another embodiment, the outer surface is cationic at physiological pH. In a related embodiment, the charged outer surface is comprised of polyethylene glycol (PEG). In a related embodiment, the PEG is derivitized, e.g., to comprise a trimethyl ammonium moiety, a carboxylic acid moiety, a sulfonic acid moiety, or a hydroxyl group.

In a related embodiment, the nanostructure has a net anionic charge at physiological pH subsequent to cleavage by the protease.

In another aspect, the invention provides a nanostructure for delivering a cancer therapeutic or an imaging agent to a tumor comprising a cleavable outer surface and an inner core containing the cancer therapeutic or the imaging agent, wherein the outer surface is attached to the inner core by peptide linkers, and the cleavable outer surface is cleaved when the nanostructure is proximate to or within the tumor.

In a related embodiment, the peptide linkers have the amino acid sequence PLGVRG (SEQ ID NO:1) or PLGLAG (SEQ ID NO:2). In another embodiment, the nanostructure is cationic prior to cleavage and anionic subsequent to cleavage.

In another aspect, the invention provides a method of treating or diagnosing a subject with a tumor comprising administering to the subject a nanostructure comprising a charged outer surface and an inner core comprising a cancer therapeutic or an imaging agent, wherein the charged outer surface and the inner core are connected by peptides which are cleaved by a protease in the tumor, and wherein the charged outer surface provides effective delivery of the nanostructure to a tumor, and the size and/or charge of the inner core provides effective delivery within the tumor.

In a related embodiment, the inner core further comprises one or more quantum dots, e.g. CdSe quantum dots. In another related embodiment, the charged outer surface is attached to the inner core by a peptide, e.g., a peptide that is cleavable by a protease expressed by a tumor cell such as Cathepsin B, Cathepsin D, MMP-2, Cathepsin K, Prostate-specific antigen, Herpes simplex virus protease, cytomegalovirus protease, thrombin, or interleukin 1β converting enzyme. In a specific embodiment, the peptide is a MMP-2 cleavable peptide and has the sequence PLGVRG (SEQ ID NO:1) or PLGLAG (SEQ ID NO:2).

In a related embodiment, the charged outer surface is comprised of polyethylene glycol (PEG). In a related embodiment, the PEG is derivitized, e.g., the PEG is derivatized to comprise a trimethyl ammonium moiety, a carboxylic acid moiety, a sulfonic acid moiety, or a hydroxyl group.

In another embodiment, the nanostructure is about 10-30 nm in diameter, about 10-50 nm in diameter, about 10-100 nm in diameter, or about 10-400 nm in diameter.

In another embodiment, the inner core comprises a matrix of PEG silicate.

In another embodiment, the nanostructure has a charge of about −80 mv to about 60 mv.

In another embodiment, the charged outer surface of the nanostructure is cationic. In another embodiment, the nanostructure is cationic prior to cleavage and anionic subsequent to cleavage of the outer surface.

In another aspect, the instant invention provides a method of treating cancer in a subject comprising administering to the subject a therapeutically effective amount of a nanostructure of the invention, thereby treating the cancer in the subject.

In another aspect, the instant invention provides a method of inhibiting the growth of a tumor in a subject comprising administering to the subject a therapeutically effective amount of a nanostructure of the invention, thereby inhibiting the growth of the tumor in the subject.

In another embodiment, the method of the invention further comprises the step of obtaining the nanostructure of the invention prior to administering it to a subject.

In another aspect, the instant invention provides a pharmaceutical composition comprising a nanostructure of the invention and a pharmaceutically acceptable carrier.

In another embodiment, the instant invention provides a kit comprising a nanostructure of the invention and instructions for use according to the methods herein.

Other aspects of the invention are described in or are obvious from the following disclosure, and are within the ambit of the invention.

### DETAILED DESCRIPTION

Intravascularly injectable nanostructures are a class of nano-devices or “nano-vectors” which have been proposed for use against cancer (Ferrari, M. (2005) Nature Reviews Cancer, 5: 161-171). Multifunctionality is the fundamental advantage of nanopaiticles for the cancer-specific delivery of therapeutic and imaging agents and formulations can be designed to extend circulation time (e.g. with PEG), to protect active agents from enzymatic or environmental degradation, to increase the amount or number of payloads, to overcome physiological barriers and to target specific moieties in tumors (Duncan, R. (2003) Nature Rev. Drug Discov., 2: 347-360; Allen, T. M (2002) Nature Rev. Cancer., 2: 750-763; and Ferrari, M. (2005) Nature Reviews Cancer, 5: 161-171; Torchilin, V. (2005) Nat Rev Drug Discov. 4:145-60.

Although targeting tumors by biomarker recognition (such as antibody- or receptor-mediated delivery) is being extensively studied, nano-vectors having optimized physico-chemical properties, such as size and charge, may better determine the efficacy of any therapeutic or diagnostic approach.

Although increasing the size of a nano-vector allows it to incorporate larger payloads, larger particles often have impaired transport through physiological barriers such as the vessel wall and interstitium. Recent studies have demonstrated that tumor vessels have a pore cut-off size to nanostructures that is relatively large compared to that of normal vessels (Hobbs et al. (1998) P.N.A.S. 95: 4607-4612; Yuan, F. et al. (1995) Cancer Research, 55:3752-3756). Taking advantage of this difference between tumor and normal vessels, relatively large nanostructures can be selectively delivered to tumors. However, after crossing the endothelium, large nanostructures are not able to penetrate the tumor interstitium (Jain, R. K. Nature Medicine, 4: 655-657).

In tumors, vascular endothelial cells display anionic phospholipids such as phosphatidylserine in the outer lipid layer; these are restricted to internal bilayer surface in most normal endothelial cells (Ran, S. et al. (2002) Cancer Research, 62: 6132-6140; Ran, S. and Thorpe, P. E. (2002) Int J Radiat Oncol Biol Phys, 54: 1479-1484). This indicates that it is possible to selectively target tumor vessels with cationic molecules. In addition, many basement membrane proteoglycans such as heparan sulfate are negatively charged, making the basement membrane highly anionic. It has been demonstrated that cationic macromolecules and liposomes accumulate in tumor vessels more efficiently than neutral or anionic molecules (Dellian et al. (2000) Br. J. Cancer 82:1513-8); Thurston et al. (1998) J. Clin. Invest. 101:1401-1413). However, it has been shown that interstitial accumulation of anionic photosensitizer conjugates is more efficient than their cationic counterparts (Hamblin, M. et al. (1999) Br. J. Cancer, 81: 261-268). Based on these findings, cationic particles prefer to localize at the basement membrane, while anionic particles are better suited for interstitial transport.

Accordingly, the instant invention is based, at least in part, on the observation that larger nanostructures (e.g., larger than 100 nm) and nanostructures with higher cationic charge will preferentially target tumor vasculature compared to normal vessels (see, for example, Campbell, R. B. et al. (2002) Cancer Research, 62: 6831-6836; Dellian, M. (2000) Br. J. Cancer 82: 1513-1518; Hobbs, S. K., et al. (1998) PNAS 95: 4607-4612; Krasnici, S. (2003) Int J Cancer 105: 561-567; Thurston, G., (1998) J Clin Invest 101:1401-1413; and Yuan, F., et al. (1995) Cancer Research 55:3752-3756). Further, once the nanostructures extravasate, internal transport strongly depends on size (see, for example, Pluen, A., Boucher, Y., Ramanujan, S. et al. (2001) PNAS 98: 4628-4633 and Alexandrakis, G. et al. (2004) Nature Medicine, 10: 203-207). In addition, high cationic charge will hinder their movement by interacting with negatively charged moieties of the extracellular matrix and the cell surface (see, for example, Halfter, W. et al. (1998) Journal of Biological Chemistry 273: 25404-25412; Nerlich, A. et al. (1995) Veroffentlichungen aus der Pathologie, 145:1-139; and Seno, S., et al. (1983) Annals of the New York Academy of Sciences, 416: 410-425). Accordingly, based on these studies, a nanostructure that is capable of changing size and/or charge would be useful for the delivery of therapeutic or diagnostic agents to a tumor.

### DEFINITIONS

The term “nanostructure” as used herein is intended to include multi-layered compositions for the delivery of therapeutic or diagnostic agents to a solid tumor that are not larger than about 300-400 nm in diameter. In certain embodiments, the nanostructure comprises an inner core comprised of, for example, one or more quantum dots, or a polymeric substance, comprising a diagnostic or therapeutic agent. In further embodiments, the nanostructures of the invention have an inner core surrounded by a charged outer surface. Moreover, in certain embodiments, the nanostructures of the invention are capable of changing size and/or charge.

The term “selectively removable” as used herein is directed to the property of the nanostructures of the invention whereby the charged outer layer of the nanostructure is removable under desired conditions. In certain embodiments, the outer layer is removed by cleavage of a cleavable peptide, e.g., by a protease, when the nanostructure is in a given environment, e.g., proximate to the surface of or within a solid tumor having the protease. In other embodiments, the peptide linker connecting the inner core and the outer layer is pH labile and is cleaved when the nanostructure is in an acidic environment. In another embodiment, the peptide linker is photocleavable, e.g., by UV light. The selective removal allows for decreased size and/or a change in charge of the resulting nanostructure and, therefore, improved internal transport properties.

The term “charged outer surface” is intended to mean a layer on the outside of a nanostructure that is attached, either directly or indirectly, to the core, e.g., the quantum dot or polymeric core, of a nanostructure and is selectively removable. In certain embodiments, this layer is positively charged. In other embodiments, this layer is negatively charged. The charged layer facilitates the transport of the nanostructure to the location of a tumor. Once at this location, the charged outer layer is selectively removable so as to facilitate transport of the nanostructure into the tumor.

The term “cleavable linker” is intended to mean an amino acid sequence (e.g., a peptide) that can be cleaved by an enzyme, such as a protease.

Other definitions appear in context throughout this disclosure.

### Nanostructures

The instant invention provides nanostructures that are useful in the treatment and/or diagnosis of disease, e.g., cell proliferative diseases such as cancer. The term nanostructure refers to a composition that measures less than about 300-400 nm in diameter, for example about 50 nm, about 75 nm, about 100 nm, about 150 nm, about 200 nm or about 250 nm in diameter. In specific embodiments, the nanostructures of the invention are useful in the treatment and diagnosis of solid tumors. The nanostructures of the invention have the ability to change size and/or charge, thereby leading to increased transport and delivery to the location of a tumor and, therefore, increased efficacy to a subject. For example, in one embodiment the nanostructures of the invention have a charged outer surface that can be selectively removed from an inner core that contains a cancer therapeutic or diagnostic agent. The removal of the charged outer surface allows for the nanostructure to change charge and/or size depending on the composition of the particular surface.

In certain embodiments of the invention, the nanostructure comprises a charged outer surface. This charged outer surface may be comprised of peptides, carbohydrates, polymers, or small molecules that are charged, e.g., negatively or positively charged, at physiological pH. Exemplary outer surface molecules include, but are not limited to, polyethylene glycol (PEG), N-(2-hydroxypropyl)methacrylamide (HPMA), poly(vinyl-pyrrolidone) (PVP), poly(ethyleneimine)(PEI), polyamidoamines, divinyl ether and maleic anhydride (DIVEMA), dextran (α-1,6 polyglucose, dextrin (α-1,4 polyglucose), hyaluronic acid, chitosans, polyamino acids, e.g., poly (lysine) or poly (glutamic acid), poly (malic acid), poly(sapartamides), poly co-polymers, e.g., copaxone. In specific embodiments, the charged outer surface is comprised of PEG molecules. The surface groups which comprise the charged outer surface of the nanostructure can be further functionalized and bioconjugated. For example to expose a cationic surface consisting of tri-methyl ammonium end groups, an anionic surface consisting of carboxylic acid or sulfonic acid end groups, zwiterionic by exposing an amino acid, or neutral by exposing hydroxyl groups. Albumin can be conjugated to the dots as a standard platform for further conjugation and so take advantage of the extensive knowledge available regarding albumin as a conjugation scaffold for attached proteins, antibodies, or other fluorophores. For example, Tat protein (for cellular uptake) and protease cleavable peptides can be conjugated onto albumin or directly to the ends of, for example, PEG groups.

The charged outerlayer of the nanostructure is selectively removable so as to confer advantageous transport properties upon the nanostructure. For example, removal of the charged outerlayer once the nanostructure has been transported to the tumor will increase the transport of the nanostructure into the tumor. The charged outer surface can be connected to the inner core by a linker, e.g., a cleavable peptide linker, that allows for selective removal of the charged outer surface in a desired environment. In exemplary embodiments, the inner core is comprised of one or more quantum dots, polymers, liposomes, silicon, silica, dendrimers, microbubbles and nanoshells. In particular embodiments the inner core is comprised of one or more one or more of quantum dots, polymers, liposomes, silicon, silica, dendrimers, microbubbles and nanoshells, optionally in a matrix, e.g., a PEG silicate matrix. The inner core may optionally comprise a ligand layer comprising one or more surface ligands (e.g., organic molecules) surrounding the core. In certain embodiments of the invention, the ligand layer may be used to couple a cleavable linker to the inner core, e.g., a peptide linker as described herein, to provide a linkage to the outer layer, and/or other inner core constituents.

### Inner Core Constituents

The present invention provides nanostructures, such as nanostructures having an inner core comprising, for example, one or more quantum dots, nanoshells, microbubles, liposomes, or combinations thereof.

Quantum dots used in biological applications consist of an inorganic core, typically CdSe, that is the optically active center, an inorganic protective shell, and an organic coating designed for biological compatibility and further conjugation. In certain embodiments, the organic coating is used to conjugate a charged molecule, e.g., polyethylene glycol to the quantum dot. In a specific embodiment, the charged molecule is attached via a cleavable linker molecule. The cores are typically nearly spherical semiconductor nanostructures, ranging from about 2 to 10 nm in diameter. Core-shell quantum dots have narrow fluorescence spectra, typically about 30 nm, and quantum yields that are usually in excess of 30%. Peak positions depend both on the material and size of the quantum dot. Compared to organic dye molecules, quantum dots are particularly well suited to biological tracking, e.g., diagnostic studies, that use fluorescence as the reporter. The excitation band is very broad, requiring only that the excitation wavelength be to the blue of the emission, but the emission band is narrow and symmetric. Absorption cross sections of quantum dots can surpass those of dye molecules, especially for larger quantum dots because the distance of the extinction coefficient from the fluorescence band is proportional to the volume of the dot. For example 7.0 nm CdSe quantum dots emitting at ˜660 nm, have an extinction coefficient ranging from 1.0×106 M⁻¹ cm⁻¹ at 630 nm to 6.2×10⁶ M⁻¹ cm⁻¹ at 350 nm (Leatherdale, C. A. et al. (2002) Journal of Physical Chemistry B, 106: 7619-7622). A size series of quantum dots thus represents a family of fluorophores covering a range of emission wavelengths, that are excited with the same light source, and are ideal for multiplexed detection. The accessible range of emission colors from biologically compatible quantum dots is from about 450 nm (using CdS based quantum dots) to about 800 nm (using a combination of CdSe and CdTe based quantum dots). Furthermore, because quantum dots are inorganic solids they are significantly less susceptible to photobleaching than dye molecules, making them ideal candidates for long time tracking and single molecule imaging studies.

A quantum dot will typically be in a size range between about 1 nm and about 1000 nm in diameter or any integer or fraction of an integer therebetween. Preferably, the size will be between about 1 nm and about 100 nm, more preferably between about 1 nm and about 50 nm or between about 1 nm to about 20 nm (such as about 2, 3, 4, 5, 6, 7, 8, 9, 10, 11, 12, 13, 14, 15, 16, 17, 18, 19, or 20 nm or any fraction of an integer therebetween), and more preferably between about 1 nm and 10 nm.

A core of a quantum dot may comprise inorganic crystals of Group IV semiconductor materials including but not limited to Si, Ge, and C; Group II-VI semiconductor materials including but not limited to ZnS, ZnSe, ZnTe, ZnO, CdS, CdSe, CdTe, CdO, HgS, HgSe, HgTe, HgO, MgS, MgSe, MgTe, MgO, CaS, CaSe, CaTe, CaO, SrS, SrSe, SrTe, SrO, BaS, BaSe, BaTe, and BaO; Group III-V semiconductor materials including but not limited to AlN, AlP, AlAs, AlSb, GaN, GaP, GaAs, GaSb, InN, InP, InAs, and InSb; Group IV-VI semiconductor materials including but not limited to PbS, PbSe, PbTe, and PbO; mixtures thereof; and tertiary or alloyed compounds of any combination between or within these groups. Alternatively, or in conjunction, a core can comprise a crystalline organic material (e.g., a crystalline organic semiconductor material) or an inorganic and/or organic material in either polycrystalline or amorphous form.

A core may optionally be surrounded by a shell of a second organic or inorganic material. A shell may comprise inorganic crystals of Group IV semiconductor materials including but not limited to Si, Ge, and C; Group II-VI semiconductor materials including but not limited to ZnS, ZnSe, ZnTe, ZnO, CdS, CdSe, CdTe, CdO, HgS, HgSe, HgTe, HgO, MgS, MgSe, MgTe, MgO, CaS, CaSe, CaTe, CaO, SrS, SrSe, SrTe, SrO, BaS, BaSe, BaTe, and BaO; Group III-V semiconductor materials including but not limited to AlN, AlP, AlAs, AlSb, GaN, GaP, GaAs, GaSb, InN, InP, InAs, and InSb; mixtures thereof; and tertiary or alloyed compounds of any combination between or within these groups. Alternatively, or in conjunction, a shell can comprise a crystalline organic material (e.g., a crystalline organic semiconductor material) or an inorganic and/or organic material in either polycrystalline or amorphous form. A shell may be doped or undoped, and in the case of doped shells, the dopants may be either atomic or molecular. A shell may optionally comprise multiple materials, in which different materials are stacked on top of each other to form a multi-layered shell structure.

In at least one embodiment of the quantum dot may optionally comprise a ligand layer comprising one or more surface ligands (e.g., organic molecules) surrounding the core. In certain embodiments of the invention, the ligand layer may be used to couple a cleavable linker to the quantum dot, e.g., a peptide linker as described herein.

Quantum dots can be chemically synthesized using wet chemical techniques that have been well described in the literature. A typical preparation consists of rapidly introducing a solution consisting of a Cd precursor, such as a cadmium carboxylate salt and a Se precursor, typically trioctylphosphine selenide (TOPSe), into a hot (>300° C.) solvent mixture that contains coordinating species such as phosphonic acids, amines, and trioctylphosphine oxide (TOPO). The size of the nanocrystals obtained is precisely determined by a combination of precursor concentrations, stoichoimetric ratios, temperature, and length of reaction. Shells of ZnS or CdZnS, typically 1-2 nm thick, are grown on top of CdSe cores that have been isolated and redispersed in solutions typically consisting of mixtures of alkyl phosphines and alkyl amines. Precursors for the shell typically include diethyl zinc, dimethyl cadmium, or organic salts of Zn and Cd, and (TMS)2S. Characterization of quantum dot samples relies on Transmission Electron Microscopy (TEM) for sizing and for assessing crystal quality, UV-Vis absorption spectroscopy, and fluorescence spectroscopy for emission wavelength, linewidth, and quantum yield determination. Emission lifetimes are typically 10-25 nseconds. The spherical shape has largely been the standard for quantum dots used in biological studies, but varying this is likely a parameter that can add functionality and information. quantum dots can be grown in a variety of shapes, e.g. as nanorods with diameters <10 nm and aspect ratios as large as 10:1 or tetrapods that consist of four nanorods attached together at a central point. Varying the shape is typically achieved using combinations of alkyl phosphinic acids and by kinetically forcing the growth along the crystal axis through a large excess of precursors in solution.

For quantum dots designed for in vivo imaging or diagnostic applications, the main contributor to size is the organic coating which renders the quantum dot soluble and stable in plasma. This coating also allows peptides, e.g., peptide linker, cleavable peptides, to be covalently conjugated. The coating generally consists of an hydrophobic component that associates with the quantum dot, a hydrophilic or charged component for solubility, and a means for further conjugation (Michalet, X., et al. (2001) Single Molecules, 2: 261-276). Typically the hydrophilic component consists of PEG moieties to minimize non-specific binding and increased vascular circulation times. The important functions of the quantum dot coating are to prevent degradation of its chemical and optical properties and provide stability against agglomeration. The protective role of the organic coating is maximized by using molecules that can be cross-linked to each other, before or after their association to the quantum dot surface, to form what is effectively a poly-dentate coating unlikely to leave the quantum dot surface (through the usual dynamic binding and un-binding events typical of quantum dot-capping group associations). Further conjugation can be designed to be through the ends of the PEG chains for better accessibility, or closer to the quantum dot.

Examples of coatings for biocompatible quantum dots include organo-silica shells in which silica provides cross-linking and serves as a platform for further conjugation, ambiphilic polymers that associate hydrophobically with the native organic groups (usually TOPO) on the surface of as grown quantum dots, dendrimers, and oligomeric phosphines. Other approaches include the use of electrostatic interactions to form encapsulated particles, or the interdigitation of hydrocarbon chains, for example by using phospholipids (Dubertret, B., et al. (2002) Science, 298: 1759-1762). In all cases, conjugation to biomolecules for selective targeting is usually achieved through well-known bioconjugation techniques, such as EDC coupling with N-hydroxysuccinimides. Quantum dots can be made soluble in plasma using one of three established approaches (1) an ambiphilic polymer consisting of an acrylic acid backbone functionalized with alkyl side chains, (2) phospholipids, and (3) oligomeric phosphines that provide multiple attachment points to the quantum dot surface and expose carboxylic acid functional groups for further water compatibility and further conjugation. In all three approaches, PEGylation with varying size PEG chains allows tuning of the hydrodynamic size from 10 to 30 nm.

Clusters of quantum dots can be formed using peptide linkers. These clusters can be built so that upon cleavage by a protease, clusters break apart into individual nanostructures, so providing the possibility of decreasing the size of the probe upon exposure to the tumor environment. Clusters of quantum dots can be synthesized with two colors so as to be able to observe the break-up in vivo through a sudden change in the spectrum of the probe, or at the single dot level the disappearance of one of the two colors. If the quantum dots are close enough for efficient energy transfer within the clusters, it is likely that the redder of the two colors will dominate while the cluster is intact, with both colors observed upon break-up. This can be monitored with in vitro FRET characterization experiments.

The inner core may also contain one or more nanoshells, e.g., metal nanoshells. Nanoshells are nanoparticles comprised of a dielectric core surrounded by an ultra-thin metal and characterized by highly tunable optical resonances (Hirsch et al. (2006) Annals of Biomedical Engineering 34:15-22). Nanoshells have been shown to effectively kill tumor cells when used in conjunction with near-infrared light (Hirsch et al. (2003) PNAS 100:13549-13554).

The inner core can also contain one or more microbubbles. Microbubbles comprise a water insoluble gas surrounded by a biological layer, e.g., a lipid layer. Synthetic microbubbles have been developed and are useful imaging agents due to their altered reflectivity of ultrasound energy (Feinstein et al. (2004) Am J Phsyiol Heart Circ Physiol. H450-7). In addition to the ability of microbubbles to be used as imaging agents, they can also be used to deliver therapeutics by, for example, conjugating biological or chemical moieties to the biological layer (Klibanov et al. (2006) Investigative Radiology 41:354-62.

The inner core can contain one or more liposomes. Liposomes are microscopic phospholipid bubbles with a bilayerd membrane that have been shown to be effective for delivering therapeutic and imaging agents (for a review see, Torchilin, V. (2005) Drug Discovery 4:145-160). The half-life of liposomes can be extended by protecting them with, for example, polyethylene glycol, poly[N-(2-hydroxylpropyl)methacrylamide], poly-N-vinylpyrrolidones, L-amino-acid-based biodegradable polymer-lipid conjugates or polyvinyl alcohol, thus increasing their utility for the delivery of therapeutic or imaging agents. A number of liposomes are currently marketed. For example, Doxil® is a pegylated Liposomal composition containing doxorubicin used for the treatment of cancer (Gabizon et al. (2003) Clin. Pharmacokinet 42:419-36).

The inner core materials of the invention disclosed herein, e.g., therapeutic and imaging agents, can be combined within a single nanostructure to provide beneficial treatment or diagnostic effects. For example, the nanostructures of the invention may contain one or more anticancer agents, i.e., cancer therapeutic agents. The anticancer agents may be formulated into the inner core of the nanostructure, e.g., into a polymer matrix. Alternatively, the therapeutic agent may be formulated into material that is used to formulate the inner core of a nanostructure comprising multiple inner core constituents (e.g., quantum dots). In another embodiment, the anticancer agent may be conjugated to the coating of the inner core, e.g., of a quantum dot, so as to be covalently, or non-covalently attached to the inner core after cleavage of the charged layer.

Exemplary cancer therapeutic agents include chemical or biological reagents that inhibit the growth of proliferating cells or tissues wherein the growth of such cells or tissues is undesirable. Chemotherapeutic agents are well known in the art (see e.g., Gilman A. G., et al., The Pharmacological Basis of Therapeutics, 8th Ed., Sec 12:1202-1263 (1990) and Teicher, B. A. Cancer Therapeutics: Experimental and Clinical Agents (1996) Humana Press, Totowa, N.J.), and are typically used to treat neoplastic diseases. Other similar examples of chemotherapeutic agents include: bleomycin, docetaxel (Taxotere), doxorubicin, edatrexate, erlotinib (Tarceva), etoposide, finasteride (Proscar), flutamide (Eulexin), gemcitabine (Gemzar), genitinib (Irresa), goserelin acetate (Zoladex), granisetron (Kytril), imatinib (Gleevec), irinotecan (Campto/Camptosar), ondansetron (Zofran), paclitaxel (Taxol), pegaspargase (Oncaspar), pilocarpine hydrochloride (Salagen), porfimer sodium (Photofrin), interleukin-2 (Proleukin), rituximab (Rituxan), topotecan (Hycamtin), trastuzumab (Herceptin), tretinoin (Retin-A), Triapine, vincristine, and vinorelbine tartrate (Navelbine).

In alternative embodiment, the nanostructure may be used to deliver an imaging agent, i.e., a detectable label, to a tumor. The detectable label can be directly detectable (i.e., one that emits a signal itself). Alternatively, the detectable label can be indirectly detectable (i.e., one that binds to or recruits another molecule that is itself directly detectable, or one that cleaves a product to generate directly detectable substrates). Generally, the detectable label can be selected from the group consisting of an electron spin resonance molecule (such as for example nitroxyl radicals), a fluorescent molecule, a chemiluminescent molecule, a radioisotope, an enzyme, an enzyme substrate, a biotin molecule, an avidin molecule, a streptavidin molecule, a peptide, an electrical charge transferring molecule, a colloid gold nanocrystal, a ligand, a microbead, a magnetic bead, a paramagnetic particle, a chromogenic substrate, an affinity molecule, a protein, a peptide, a nucleic acid, a carbohydrate, an antigen, a hapten, an antibody, an antibody fragment, and a lipid. In specific embodiments, gadolinium, manganese and iron may be used as detectable labels for MRI; iodine may be used for X-ray/CT; and low density lipids and gas microbubbles in a stabilizing shell for ultrasound.

The surface of the inner core can be conjugated with a cleavable peptide linker, e.g., a protease cleavable linker, that provides a linkage to the outer layer or to other inner core constituents. Exemplary proteases useful in the methods of the invention include, but are not limited to: Cathepsin B, Cathepsin D, MMP-2, Cathepsin K, Prostate-specific antigen, Herpes simplex virus protease, HIV protease, cytomegalovirus protease, thrombin, and interleukin 1β converting enzyme. Moreover, peptides that comprise the protease recognition sequence of theses proteases are useful in the methods and compositions of the invention (for details regarding the sequence of protease cleavable linkers see, for example, Mahmood et al. (2003) Molecular Cancer Therapeutics 2:489-96). In specific embodiments, linkers useful in the methods of the invention include thrombin cleavable linkers (see, ChemBioChem 3:207-211, 2002), Cathepsin cleavable linkers (see, Bioconjugate Chem 9: 618-626), MMP-2 cleavable linker (see JBC 265: 20409-20413, 1990), HIV protease cleavable linker (see, Bioorganicheskaia Khimiia 25:911-922, 1999), acid cleavable linkers (see, Crit. Rev Drug Carrier Syst 16:245-288, 1999), and photo cleavable linkers, e.g., those available from Novabiochem).

The peptide linker can itself be conjugated to render it, for example, cationic (with trimethyl ammonium or anionic with carboxylic acid or sulfonic acid). Upon cleavage, the particles can then switch potential from cationic to anionic or vice versa as follows. The cleavable peptide sequence can be coupled to terminal amine or to carboxylic acid groups using established conjugation chemistries. Unconjugated carboxylic acid (amine) groups provide negative (positive) charge which is balanced by the cationic (ionic) charge conjugated to the peptide. Upon cleavage, the net charge of the nanostructures switches from cationic to anionic or vice versa. The peptide linker can also be functionalized with a fluorescent dye, such as a rhodamine based conjugate. In specific embodiments, the quantum dot can serve as an efficient FRET acceptor if the dye emission overlaps with the absorption of the quantum dot, and, if the two are close enough. Upon cleavage, fluorescence from the dye will be observed if FRET is efficient. If FRET is not efficient, the quantum dot-linker-dye complex will co-localize emission from the two colors, while upon cleavage the position of the two colors will be distinct. In vitro FRET control experiments will be used to characterize these complexes.

### Methods of Treatment

The term “subject” is intended to include organisms, e.g., prokaryotes and eukaryotes, which are capable of suffering from or afflicted with a cell proliferative disorder, e.g., cancer. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from cancer.

The term “neoplasia” or “neoplastic transformation” is the pathologic process that results in the formation and growth of a neoplasm, tissue mass, or tumor. Such process includes uncontrolled cell growth, including either benign or malignant tumors. Neoplasms include abnormal masses of tissue, the growth of which exceeds and is uncoordinated with that of the normal tissues and persists in the same excessive manner after cessation of the stimuli that evoked the change. Neoplasms may show a partial or complete lack of structural organization and functional coordination with the normal tissue, and usually form a distinct mass of tissue. One cause of neoplasia is dysregulation of the cell cycle machinery.

Neoplasms tend to morphologically and functionally resemble the tissue from which they originated. For example, neoplasms arising within the islet tissue of the pancreas resemble the islet tissue, contain secretory granules, and secrete insulin. Clinical features of a neoplasm may result from the function of the tissue from which it originated. For example, excessive amounts of insulin can be produced by islet cell neoplasms resulting in hypoglycemia which, in turn, results in headaches and dizziness. However, some neoplasms show little morphological or functional resemblance to the tissue from which they originated. Some neoplasms result in such non-specific systemic effects as cachexia, increased susceptibility to infection, and fever.

By assessing the histology and other features of a neoplasm, it can be determined whether the neoplasm is benign or malignant. Invasion and metastasis (the spread of the neoplasm to distant sites) are definitive attributes of malignancy.

Despite the fact that benign neoplasms may attain enormous size, they remain discrete and distinct from the adjacent non-neoplastic tissue. Benign tumors are generally well circumscribed and round, have a capsule, and have a grey or white color, and a uniform texture. In contrast, malignant tumors generally have fingerlike projections, irregular margins, are not circumscribed, and have a variable color and texture. Benign tumors grow by pushing on adjacent tissue as they grow. As the benign tumor enlarges it compresses adjacent tissue, sometimes causing atrophy. The junction between a benign tumor and surrounding tissue, may be converted to a fibrous connective tissue capsule allowing for easy surgical removal of the benign tumor.

Conversely, malignant tumors are locally invasive and grow into the adjacent tissues usually giving rise to irregular margins that are not encapsulated making it necessary to remove a wide margin of normal tissue for the surgical removal of malignant tumors. Benign neoplasms tend to grow more slowly and tend to be less autonomous than malignant tumors. Benign neoplasms tend to closely histologically resemble the tissue from which they originated. More highly differentiated cancers, i.e., cancers that resemble the tissue from which they originated, tend to have a better prognosis than poorly differentiated cancers, while malignant tumors are more likely than benign tumors to have an aberrant function, e.g., the secretion of abnormal or excessive quantities of hormones.

The term “cancer” includes malignancies characterized by deregulated or uncontrolled cell growth, for instance carcinomas, sarcomas, leukemias, and lymphomas. The term “cancer” includes primary malignant tumors, e.g., those whose cells have not migrated to sites in the subject's body other than the site of the original tumor, and secondary malignant tumors, e.g., those arising from metastasis, the migration of tumor cells to secondary sites that are different from the site of the original tumor.

The term “carcinoma” includes malignancies of epithelial or endocrine tissues, including respiratory system carcinomas, gastrointestinal system carcinomas, genitourinary system carcinomas, testicular carcinomas, breast carcinomas, prostate carcinomas, endocrine system carcinomas, melanomas, choriocarcinoma, and carcinomas of the cervix, lung, head and neck, colon, and ovary. The term “carcinoma” also includes carcinosarcomas, which include malignant tumors composed of carcinomatous and sarcomatous tissues. The term “adenocarcinoma” includes carcinomas derived from glandular tissue or a tumor in which the tumor cells form recognizable glandular structures.

The term “sarcoma” includes malignant tumors of mesodermal connective tissue, e.g., tumors of bone, fat, and cartilage.

For example, the therapeutic methods of the present invention can be applied to cancerous cells of mesenchymal origin, such as those producing sarcomas (e.g., fibrosarcoma, myxosarcoma, liosarcoma, chondrosarcoma, osteogenic sarcoma or chordosarcoma, angiosarcoma, endotheliosardcoma, lympangiosarcoma, synoviosarcoma or mesothelisosarcoma); leukemias and lymphomas such as granulocytic leukemia, monocytic leukemia, lymphocytic leukemia, malignant lymphoma, plasmocytoma, reticulum cell sarcoma, or Hodgkin's disease; sarcomas such as leiomysarcoma or rhabdomysarcoma, tumors of epithelial origin such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary-carcinoma, transitional cell carcinoma, chorioaencinoma, semonoma, or embryonal carcinoma; and tumors of the nervous system including gioma, menigoma, medulloblastoma, schwannoma or epidymoma. Additional cell types amenable to treatment according to the methods described herein include those giving rise to mammary carcinomas, gastrointestinal carcinoma, such as colonic carcinomas, bladder carcinoma, prostate carcinoma, and squamous cell carcinoma of the neck and head region. Examples of cancers amenable to treatment according to the methods described herein include vaginal, cervical, and breast cancers.

The language “inhibiting growth,” as used herein, is intended to include the inhibition of undesirable or inappropriate cell growth. The inhibition is intended to include inhibition of proliferation including rapid proliferation. For example, the cell growth can result in benign masses or the inhibition of cell growth resulting in malignant tumors. Examples of benign conditions which result from inappropriate cell growth or angiogenesis are diabetic retinopathy, retrolental fibrioplasia, neovascular glaucoma, psoriasis, angiofibromas, rheumatoid arthritis, hemangiomas, Karposi's sarcoma, and other conditions or dysfunctions characterized by dysregulated endothelial cell division.

The language “inhibiting tumor growth” or “inhibiting neoplasia” includes the prevention of the growth of a tumor in a subject or a reduction in the growth of a pre-existing tumor in a subject. The inhibition also can be the inhibition of the metastasis of a tumor from one site to another. In particular, the language “tumor” is intended to encompass both in vitro and in vivo tumors that form in any organ or body part of the subject. Examples of the types of tumors intended to be encompassed by the present invention include those tumors associated with breast cancer, skin cancer, bone cancer, prostate cancer, liver cancer, lung cancer, brain cancer, cancer of the larynx, gallbladder, esophagus, pancreas, rectum, parathyroid, thyroid, adrenal, neural tissue, head and neck, colon, stomach, bronchi, kidneys. Specifically, the tumors whose growth rate is inhibited by the present invention include basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglioneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas (i.e. maglinant lymphomas, mantle cell lymphoma), malignant melanomas, multiple myeloma, epidermoid carcinomas, and other carcinomas and sarcomas.

The language “chemotherapeutic agent” includes chemical reagents that inhibit the growth of proliferating cells or tissues wherein the growth of such cells or tissues is undesirable. Chemotherapeutic agents are well known in the art (see e.g., Gilman A. G., et al., The Pharmacological Basis of Therapeutics, 8th Ed., Sec 12:1202-1263 (1990)), and Teicher, B. A. Cancer Therapeutics: Experimental and Clinical Agents (1996) Humana Press, Totowa, N.J. Other similar examples of chemotherapeutic agents include: bleomycin, docetaxel (Taxotere), doxorubicin, edatrexate, erlotinib (Tarceva), etoposide, finasteride (Proscar), flutamide (Eulexin), gemcitabine (Gemzar), genitinib (Irresa), goserelin acetate (Zoladex), granisetron (Kytril), imatinib (Gleevec), irinotecan (Campto/Camptosar), ondansetron (Zofran), paclitaxel (Taxol), pegaspargase (Oncaspar), pilocarpine hydrochloride (Salagen), porfimer sodium (Photofrin), interleukin-2 (Proleukin), rituximab (Rituxan), topotecan (Hycamtin), trastuzumab (Herceptin), tretinoin (Retin-A), Triapine, vincristine, and vinorelbine tartrate (Navelbine).

The language “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier should be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

The instant invention provides therapeutic methods for the treatment of a subject having cancer. In certain embodiments, a subject is administered a nanostructure of the invention to alleviate one or more symptoms of cancer. For example, a subject can be administered a nanostructure of the invention to reduce the size or eliminate a solid tumor.

The dosages administered will vary from patient to patient; a “therapeutically effective dose” can be determined, for example, by monitoring the size or growth rate, or the duration of the growth period of a tumor, tumor number, cancer cell number, viability, growth rate and the duration of the growth period of a cancer cell. A therapeutically effective dose refers to a dose wherein the combination of compounds has a synergistic effect on the treatment of cancer.

In the treatment of cancer, a therapeutically effective dosage regimen should be used. By “therapeutically effective”, one refers to a treatment regimen sufficient to decrease tumor size or tumor number, decrease the rate of tumor growth or kill the tumor. Alternatively, a “therapeutically effective regimen” may be sufficient to arrest or otherwise ameliorate symptoms of the cancer. Generally, in the treatment of cancer, an effective dosage regimen requires providing the medication over a period of time to achieve noticeable therapeutic effects. The pharmaceutical composition may be formulated from a range of preferred doses, as necessitated by the condition of the patient being treated.

The present invention is additionally described by way of the following illustrative, non-limiting Examples that provide a better understanding of the present invention and of its many advantages.

### EXAMPLES

Preparation of Quantum Dots: Quantum Dots Will be Chemically Synthesized Using wet chemical techniques that have been well described in the literature. A typical preparation will consist of rapidly introducing a solution consisting of a Cd precursor, such as a cadmium carboxylate salt and a Se precursor, typically trioctylphosphine selenide (TOPSe), into a hot (>300° C.) solvent mixture that contains coordinating species such as phosphonic acids, amines, and trioctylphosphine oxide (TOPO). The size of the nanocrystals obtained is precisely determined by a combination of precursor concentrations, stoichoimetric ratios, temperature, and length of reaction. Shells of ZnS or CdZnS, typically 1-2 nm thick, are grown on top of CdSe cores that have been isolated and redispersed in solutions typically consisting of mixtures of alkyl phosphines and alkyl amines. Precursors for the shell typically include diethyl zinc, dimethyl cadmium, or organic salts of Zn and Cd, and (TMS)2S. Characterization of quantum dot samples relies on Transmission Electron Microscopy (TEM) for sizing and for assessing crystal quality, UV-Vis absorption spectroscopy, and fluorescence spectroscopy for emission wavelength, linewidth, and quantum yield determination. Emission lifetimes are typically 10-25 nseconds.

The spherical shape has largely been the standard for quantum dots used in biological studies, but varying this is likely a parameter that can add functionality and information. Quantum dots can be grown in a variety of shapes, e.g. as nanorods with diameters <10 nm and aspect ratios as large as 10:1 or tetrapods that consist of four nanorods attached together at a central point. Varying the shape is typically achieved using combinations of alkyl phosphinic acids and by kinetically forcing the growth along the crystal axis through a large excess of precursors in solution.

Surface Coating of Quantum dot-Silica Nanostructure Composites: The quantum dot-silica spheres will be PEGylated using well developed silica chemistry that has been previously described. This results in submicron fluorescent probes that are bio-compatible, with long vascular circulation times, and with the possibility of further conjugation for targeting or changing surface zetapotentials. Further conjugation can be designed to be through the ends of the PEG chains for better accessibility, or closer to the silica sphere. This choice depends on the application. Unlike the smaller quantum dots, the hydrodynamic size of these larger silica spheres is largely determined by the size sphere itself, with the PEG groups adding at up to about <10 nm to their diameters. Conjugations schemes described above for the quantum dots with protease cleavable proteins can be used with these nanostructures.

Characterization of Coated Quantum dots: All quantum dot preparations will be characterized by (1) gel-filtration chromatography using globular protein standards, so that an effective HD is known prior to experimentation and (2) zeta-potential measurements using a zeta-potential analyzer to probe surface charge.

Biodistribution Assays: Biodistribution of quantum dots will be gauged by conjugating dots with DOTA (1,4,7,10-tetraazacyclododecane-1,4,7,10-tetraacetic acid), followed by exposure to a standard radionucleotide, as this is a sensitive method of gauging biodistribution. Biodistribution can also be analyzed by direct chemical analysis using ICP (Inductively Coupled Plasma) emission spectroscopy, a standard analytical tool, to detect the concentration of Cd.

Quantum dot-silica nanostructure composites: The high photostability, good fluorescence efficiency and wide emission tunability of colloidally synthesized quantum dots make them excellent choices as chromophores for incorporation in such sub-micron spheres. We have developed a facile procedure of incorporating coreshell CdSe/ZnS and CdS/ZnS quantum dots into monodisperse silica microspheres. The applicability of microspheres for in vivo imaging has already been demonstrated. Microspheres of two distinct diameters were labeled with quantum dots of different emission wavelengths and administered via tail vein injection to a mouse bearing a cranial window and expressing the green fluorescent protein in vascular endothelial cells (Tie2-GFP mouse). Circulating microspheres, coated with PEG to lengthen residence times in the vessels, were imaged via multiphoton microscopy (MPM) using 800 nm light delivered through a 20×, 0.9 NA water-immersion lens. Using MPM intravitally, circulating microspheres could be tracked. The ability to track distinct microspheres of multiple well-defined sizes and colors provides simultaneous information regarding flow characteristics in blood vessels, and will be used to further optimize drug delivery strategies. High monodispersity allows similar sized spheres (400 nm and 500 nm diameters, for example) with different emission wavelengths to be used and reliably distinguished in biological experiments. Moreover, the favorable optical properties of high-quality quantum dot—simultaneous excitation of different-colors, narrow emission profiles, and high photostability—will be harnessed for biological imaging applications inaccessible to organic dye-doped submicron spheres.

Preparation of Quantum Dot-Silica Nanostructure Composites: the Procedure for incorporating CdS/ZnS and CdSe/ZnS core-shell quantum dot into submicron silica spheres, consists of incorporating the quantum dot in shells grown on preformed silica microspheres. These microsphere composites uniquely combine the following features: (1) inherent size monodispersity due to the separate microsphere core synthesis, (2) the maintenance of narrow emission profiles, (3) the silica surface is available for easy derivation with PEG groups and further conjugation. Composites in size ranging from 100 to >500 nm have already been demonstrated and the range from 30-100 nm will be developed. Analogous to the method of Vrij et al., our strategy employs growing a shell of silica onto a silica microsphere in the presence of properly derivatized quantum dots. The core silica microspheres are synthesized using previously established techniques. The native tri-noctylphosphine oxide (TOPO) ligands on the quantum dot surface are exchanged with 5-amino-1-pentanol (AP) and 3-aminopropyltrimethoxysilane (APS). The amino group of each ligand binds to the nanocrystal surface and the hydroxyl group of the AP permits dispersion in ethanol while the alkoxysilane moiety of the APS allows the formation of siloxane bonds with the silica host matrix. The cap-exchanged quantum dots are then dispersed in a mixture of ethanol, tetraethoxysilane and silica microspheres. Addition of water and ammonium hydroxide to this mixture at elevated temperatures causes rapid hydrolysis of the siloxane precursor, which subsequently condenses to form a thin shell of silica around the microsphere.

Characterization of Quantum dot-Silica Nanostructure Composites. The results of the overcoating procedure are monitored by obtaining a TEM image of a typical distribution of silica microspheres with CdSe/ZnS quantum dot localized in the shells. The size dispersities of samples are quantified by analyzing TEM images with the software package Image J. The overcoating process does not appear to be perturbed within the range of concentrations of quantum dots used. The developed overcoating process allows for the incorporation of different color-emitting quantum dots into silica microspheres of diameters ranging from 100 nm to 1,000 nm after overcoating, the loading of CdSe/ZnS NCs into the silica shell can be estimated by first acquiring the absorption spectrum of a known weight of overcoated microspheres, which yields the total number of quantum dots and microspheres, respectively. The microspheres are immersed in a refractive index matching liquid in order to minimize effects from light scattering by the microspheres. The reported density of Stöber silica microspheres is 2.03 g/cm3, with a corresponding index of refraction of 1.46. This is in agreement with the determined refractive index of our spheres. Use of this reported density allows us to quantify the number of quantum dots per microsphere. As an example, the 295 nm microspheres contain ˜1200 quantum dots per microsphere (−0.3% volume fraction). Knowing the number density of quantum dots in spheres also enables the determination the quantum yield within the sphere, and quantum yields ˜20% are readily obtained. The photostability of the quantum dots in the silica microspheres will were evaluated using a 514 nm excitation source from a CW Ar+ laser focused through a 0.95 NA air objective at an intensity of 80 W/cm2. No appreciable decrease in the fluorescence intensity was seen over a period of 20 minutes, suggesting that little, if any, photobleaching occurs.

Although the foregoing invention has been described in some detail by way of illustration and example for purposes of clarity and understanding, it will be apparent to those skilled in the art that certain changes and modifications can be practiced. Therefore, the description and examples should not be construed as limiting the scope of the invention, which is delineated by the appended numbered claims.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

1. A nanostructure comprising a charged outer surface and an inner core comprising a cancer therapeutic agent or an imaging agent, wherein the charged outer surface is selectively removable.

2. The nanostructure of claim 1, wherein the inner core is comprised of one or more members of the group consisting of quantum dots, polymers, liposomes, silicon, silica, dendrimers, microbubbles and nanoshells.

3. The nanostructure of claim 2, wherein the inner core comprises one or more quantum dots.

4. The nanostructure of claim 3, wherein the quantum dots are CdSe quantum dots.

5. The nanostructure of claim 1, wherein the charged outer surface is attached to the inner core by a peptide.

6. The nanostructure of claim 5, wherein the peptide is cleavable.

7. The nanostructure of claim 6, wherein the peptide is cleavable by a protease, by pH, or by light.

8. The nanostructure of claim 7, wherein the peptide is cleavable by a protease.

9. The nanostructure of claim 8, wherein the peptide is cleavable by a protease expressed by a tumor.

10. The nanostructure of claim 9, wherein the protease is selected from the group consisting of Cathepsin B, Cathepsin D, MMP-2, Cathepsin K, Prostate-specific antigen, Herpes simplex virus protease, HIV protease, cytomegalovirus protease, thrombin, and interleukin 1β converting enzyme.

11. The nanostructure of claim 8, wherein the protease is MMP-2.

12. The nanostructure of claim 11, wherein the cleavable peptide comprises the amino acid sequence PLGVRG (SEQ ID NO:1) or PLGLAG (SEQ ID NO:2).

13. The nanostructure of claim 1, wherein the outer surface is cationic at physiological pH.

14. The nanostructure of claim 1, wherein the charged outer surface is comprised of a material selected from the group consisting of polyethylene glycol (PEG), N-(2-hydroxypropyl)methacrylamide (HPMA), poly(vinyl-pyrrolidone) (PVP), poly(ethyleneimine) (PEI), a polyamidoamine, a mixture of divinyl ether and maleic anhydride (DIVEMA (DIVEMA), dextran (□1,6 polyglucose, dextrin (□1,4 polyglucose), hyaluronic acid, a chitosan, a polyamino acid, poly(lysine), poly(glutamic acid), poly(malic acid), poly(sapartamides), poly co-polymers, and copaxone.

15. The nanostructure of claim 14, wherein the charged outer surface is comprised of polyethylene glycol (PEG).

16. The nanostructure of claim 15, wherein the PEG is derivatized to comprise a trimethyl ammonium moiety, a carboxylic acid moiety, a sulfonic acid moiety, or a hydroxyl group.

17. The nanostructure of claim 1, wherein the nanostructure is about 10-30 nm in diameter.

18. The method of claim 1, wherein the nanostructure comprises one or more quantum dots in a matrix comprised of PEG silicate.

19. The nanostructure of claim 18, wherein the nanostructure is about 50-400 nm in diameter.

20. A nanostructure comprising a charged outer surface and an inner core comprising a cancer therapeutic agent or an imaging agent and one or more members of the group consisting of quantum dots, polymers, liposomes, silicon, silica, dendrimers, microbubbles and nanoshells, wherein the outer surface is attached to the inner core by cleavable peptides.

21. The nanostructure of claim 20, wherein the cleavable peptide is cleavable by a protease expressed by a tumor.

22. The nanostructure of claim 21, wherein the protease is selected from the group consisting of Cathepsin B, Cathepsin D, MMP-2, Cathepsin K, Prostate-specific antigen, Herpes simplex virus protease, HIV protease, cytomegalovirus protease, thrombin, and interleukin 1β converting enzyme.

23. The nanostructure of claim 22, wherein the protease is MMP-2.

24. The nanostructure of claim 23, wherein the cleavable peptide has the amino acid sequence PLGVRG (SEQ ID NO:1) or PLGLAG (SEQ ID NO:2).

25. The nanostructure of claim 20, wherein the outer surface is cationic at physiological pH.

26. The nanostructure of claim 21, wherein the nanostructure has a net anionic charge at physiological pH subsequent to cleavage by the protease.

27. A nanostructure comprising:
a cleavable outer surface; and
an inner core containing one or more cancer therapeutics or one or more imaging agents; wherein the outer surface is attached to the inner core by peptide linkers, and wherein the outer surface is cleaved when the nanostructure is proximate to or within the tumor.

28. The nanostructure of claim 27, wherein the inner core contains one or more cancer therapeutic agents.

29. The nanostructure of claim 27, wherein the inner core contains one or more imaging agents.

30. The nanostructure of claim 27, wherein the peptide linkers have the amino acid sequence PLGVRG (SEQ ID NO:1) or PLGLAG (SEQ ID NO:2).

31. The nanostructure of claim 27, wherein the nanostructure is cationic prior to cleavage and anionic subsequent to cleavage.

32. A method of delivering a cancer therapeutic or an imaging agent to a tumor in a subject comprising:
administering to the a subject nanostructure comprising:
a cleavable outer surface; and
an inner core containing one or more cancer therapeutics or one or more imaging agents; wherein the outer surface is attached to the inner core by peptide linkers, and wherein the cleavable outer surface is cleaved when the nanostructure is proximate to or within the tumor.

33. (canceled)

34. (canceled)

35. A method of treating or diagnosing a subject with a tumor comprising:
administering to the subject a nanostructure comprising a charged outer surface and an inner core comprising a cancer therapeutic or an imaging agent;
wherein the charged outer surface and the inner core are connected by peptides which are cleaved by a protease in the tumor;
and wherein the charged outer surface provides effective delivery of the nanostructure to a tumor, and the size and/or charge of the inner core provides effective delivery within the tumor.

36-51. (canceled)

52. A method of treating cancer in a subject comprising:
administering to the subject a therapeutically effective amount of the nanostructure of claim 1, thereby treating the subject.

53. A method of inhibiting the growth of a tumor in a subject comprising: administering to the subject a therapeutically effective amount of a nanostructure of claim 1, thereby inhibiting the growth of the tumor in the subject.

54. A method of delivering an imaging agent to a tumor comprising:
administering to a subject a nanostructure comprising a charged outer surface and an inner core comprising an imaging agent;
wherein the charged outer surface and the inner core are connected by peptides which are cleaved by a protease in the tumor;
and wherein the charged outer surface provides effective delivery of the nanostructure to a tumor, and the size and/or charge of the inner core provides effective delivery within the tumor.

55. (canceled)

56. A pharmaceutical composition comprising the nanostructure of claim 1 and a pharmaceutically acceptable carrier.

57. A kit comprising the nanostructure of claim 1 and instructions for use.

58. (canceled)

59. The nanostructure of claim 1, wherein the inner core is comprised of two or more members of the group consisting of quantum dots, polymers, liposomes, silicon, silica, dendrimers, microbubbles and nanoshells attached each other by a peptide.

60. The nanostructure of claim 59, wherein the peptide is cleavable.


SEQUENCE LISTING (2 sequences)

SEQ ID NO: 1 - protein, 6 aa, Artificial Sequence
  Description of Artificial Sequence Synthetic peptide
PLGVRG

SEQ ID NO: 2 - protein, 6 aa, Artificial Sequence
  Description of Artificial Sequence Synthetic peptide
PLGLAG